SEQUENCE LISTING (24 sequences)

SEQ ID NO: 1 - protein, 734 aa, unidentified
MSTTNRKKRREIKLFTLDTETRGLDGDVFRIGLFDGKQYYTGYTFADVLPVFEKYKAYDCHVYIHNLDFDLSKIIAELRDYAEPTFNNSLFINGNIVTFTASHIILHDSFRLLPSSLENLCRDFDLLEGGKMDIVDYMEENNYGIYNVKNRKLNKRLTKGNFFTTVDKDDPVLCEYMEYDCRSLYKILEIVIGLSKLEVEQFINCPTTASLAKTVYKEQYKKDYKVAISTKQYNHKQLGKGLEAFIRKGYYGGRTEVFTPRIENGYHYDKNSLYPYVMKMAEMPVGYPNVLDNEEAELSFDLWKRRRYGAGFIHAKVHVPEDMYIPILPKKDYTGKLIFPVGKIEGVWTFPELALAEAEGCKIEKIESGVVFEKTAPVFREFISYFEEIKNTSKGAKRAFSKLMQNALYGKFAMQRERIMYADISERDKLEAEGHTVSEIIYDMNGIRMEFLEYDGYAMAEYIQPHISAYITSIARILLFKGLKYAHEKGILAYCDTDSCATTTKFPDKMVHDKEYGKWKLEGYVIEGLYFQPKMYAEKAINTDGEYEEVLRMKGVPKWVVEEQLDYNSFRKWYLQVKRGKAEIPIYKGGERVQKFLTKSKNNIEMNELAEMHKTINFAREQKRNIDLNKNITSPLVRNDYGENKDEKSEYEFDEWYERLEEFNDDMNAVEELCMKFGKIQIPEKKQRKLYGLYKEYSSKAKAMCFSNEGLPIQDWCKKTGWDMKELLGELSFL

SEQ ID NO: 2 - DNA, 738 bp, unidentified
  source 1..738: mol_type = other DNA
atgaacaaaaatataaaatattctcaaaactttttaacgagtgaaaaagtactcaaccaaataataaaacaattgaatttaaaagaaaccgataccgtttacgaaattggaacaggtaaagggcattaaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 3 - DNA, 7987 bp, unidentified
  source 1..7987: mol_type = other DNA
tatgtacctctactagcctattaaaatatttacctattgacacgtaataacatttatgaaatatgatatacaaaattttctaaaattaactattgacaatttggaaaatttattggaatttccaaaaggaaaagtgtgacaagatattccaagggtgctataataggaaatgagttgaaaacgaacacgcgttttatagatgttcggtttttgtaacataactgtaacataaaaacatttgcattactggtaactttcgggtaatattggtagcagagagggacaaacgggacaaatcgagacacgatacatactctctatacaagtgaaaagaaggtggtgcatatgtaatatgagtaaactactgactgcgcaagaggttgcagacttattgcgtgttcataaaaagactatccatagaatgatacattctggaaagttagacgcttctaaggtagcaaacaaattccttattaaagaggaagatgcaaaaggactattggaaaataaaaataaaatggacaattaataggaggaatattcaatggaaaacaatactaaacaattaaataatgaggttaacgaaatcgcggtagtggacgagcatgtaaatacaaatgacatcgtaacagtggacaaggtgtcaccttcaatctttgaagcggacggagcgaaggatgtattcttctcatctattcaaagtaaagatcgtaaatcagctatcaaagtttataatgctattaactctagtgagaatgctctagcagatcatagaggtgaagtattagaaatcacggacatggttgctcatgctattactctacaagatgaattgacaaaagaagatgtggatgcattacgtgttgttttaattgataagaacggtaaagcataccacgcggtgtcacaaggggtcgtatcatctattcaaaagattattagtattgtaggtccagcaccttggaacgagcctttagaaattgttccggcagaagtgaaaacacgtaaaggatttaaaacattaactcttcaactacaaggctaatttttgtaacaaatgaatgagggtgcacattgtgtatcctcttttttaaaacataaaaggatggtgcggtcgtgaacgctattaaaaacttagttcaagtatctattcggacaaatgaacgagaactggaatttgtctcttttaatggaatggtgcatttgaagcaaaagttagaagaagatgaaacatccgtcgtgttaacaaaagaagagttagacatggttgcagctatgtctaattttgataatgtgatagccaactttatgaactctattacagaaaagaaagccatgtaagccaaagacctgtgacctagttgttacgggtcacatggctttataagtaggtgatacaatggcaaataaacggttaaagaagaaattagaaacgaaacgtaaaaagtcgttacttgtttcagaaggttatagtaagaaagaaacaaagaagctgaaaggtagagagttagaaactgtttataaaaagaaagcgcataataggaaaaatagagagcgtgctagggaaatagcaaatctagcaagacagtggggactttcaccgagtaagtttaattcatggaaaaagttacttccggaaatagagagaataaaaaaagaacaagatagggaagcaccttttctagtaatatactatcaggactttacaggtgagacagacagtaagtttatatatgattttaaaaagagaaacaatacaagatcacgtagccaaattactaggtctataataggttggttgcagaacgcccagaacaaactatttttaggaagggttgctatgcggattgtcccaaaacgtgatgtatctaaaaccaatacactttggaagaatcacggctatgtaaagatatatgagggacaaggaaaagaacttacaaaactattaacagcaatagaaacgataatggttggtgtttacgacgtaaaagaccgtgacaagtatttaaagcagttattaaataatcttcgttccttaccatataaacaagcacataggaacgctaatgagattcagaagatatatgatactaaatcatatacgaaagaaagttgggataacgatgagtactactaatagaaaaaagcgtagagagattaaactctttacgctagatactgaaacacgtggtttggacggcgacgtgtttcgaattggactattcgatggaaaacaatactatactggctatacgtttgcagacgtattaccagtgtttgaaaagtacaaagcatatgactgtcatgtatatatacataacctagacttcgacctttccaagatcatagcagagttacgtgattatgctgaaccaacatttaataactcgctatttatcaatggtaatatagtaacgtttaccgcttctcatattatactacatgatagcttccgattgctaccgtcttcacttgaaaacttatgtagagatttcgaccttttagaaggtgggaagatggatattgtcgattatatggaagaaaataattacggtatttataatgtaaaaaaccgtaaattgaacaagcgacttacaaaaggtaatttctttacaacagtagataaagatgaccctgtcctatgtgagtacatggaatatgattgtcgtagtttatataaaatcttggaaattgtaataggcctatcaaaattagaagttgaacaatttattaattgtccgactacagcttccctagctaaaacagtttataaagaacagtataagaaggattataaagttgctatttctacaaaacaatataatcataaacaactaggaaaaggtttagaggcatttattagaaaaggctactatggaggaagaaccgaagtttttacaccaagaatagaaaacggataccactatgataaaaatagtttatatccatatgttatgaaaatggctgaaatgccagtcggatacccgaatgtactggataatgaagaagcagaactttcttttgatctatggaaaagaagaagatatggagcaggctttatacatgcgaaggtacatgtacccgaagatatgtatattcctatcttacctaaaaaagactatacaggtaaattgattttcccagttggtaaaatagaaggtgtttggacatttccggaactagctttagcagaagcggaaggttgcaaaatagaaaaaatcgaaagcggtgttgtatttgagaaaacagcacctgtctttcgtgaatttatttcgtattttgaggaaataaaaaatacaagtaaaggtgctaaacgagccttttcaaaacttatgcagaatgcgttgtatggaaaatttgcaatgcagcgggaacgaatcatgtatgctgatatttctgagagagataaacttgaagcagaaggacatacagtaagcgagatcatatatgatatgaatggaattcgaatggaatttttagagtatgacggatatgctatggctgaatatattcaaccacatatttcagcatatattacttctattgcaagaattctactatttaaaggattgaagtatgcgcatgaaaaaggaattttggcttattgtgatacggacagttgtgcaacaactacgaaatttcctgataaaatggttcacgataaagagtatggaaagtggaagttagaagggtatgttattgaaggactctatttccaaccaaaaatgtatgcggaaaaagctattaataccgatggtgaatatgaggaagtattacgaatgaaaggtgttccgaaatgggttgtagaggaacaactagactacaattcatttcgtaaatggtatttacaagtaaaacgtgggaaagccgaaattcctatttataaaggcggggaacgtgttcaaaaattcttgacgaaatctaaaaataatatagaaatgaacgaactagcggaaatgcataaaacaattaattttgctagggaacaaaaaagaaacattgatctaaataaaaatattacctcacctctagttcgtaatgactatggggaaaataaggacgaaaagtcagagtatgagtttgacgaatggtatgaaagattagaagagtttaacgatgatatgaatgcggtagaggaactttgtatgaagtttggaaagatacaaataccagagaaaaagcaaaggaagttatacggactttataaagagtattcttcaaaagcgaaagctatgtgtttttcaaatgagggacttccgattcaagattggtgtaaaaagacaggatgggacatgaaagaactattaggcgaattaagtttcttataatagggagtgacgaaaaatgttgacgccaagggaacaggatacgttagaatgtataagcgggtatatggaagagtttgggtttgcaccgagtgtgcgagatatggcgagtcgaatgtacgtaagtcataagacagcacatcgctatttaatgcaattggaaagtaaaggacgaattaggagagtgcatcaaagacctagagccatccagttgttataaggaggggaaaggtatgcgtgacaaattgctcgactttatcatcgaactatcacaatctagcaaacaagtcgtaagcaagtcgtatgtcatcgacaggttaatgcaagtaacaaaagaagactacaaggaactagaaaagaaaacagaaaatgcggaaaaggactagcaaagttgctagtcttttttatttgtccaaaatgacacacgtgtgacggagcggataacaatttcacacaggaaacagctatgaccatgattacgaattcgagctcggtacccggggatcctctagagattgtaccgttcgtatagcatacattatacgaagttatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaagttaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaataacttcgtatagcatacattatacgaacggtagaatcgtcgacctgcaggcatgcaagcttggcactggccgtcgttttacaacgtcgtgactgggaaaaccctggcgttagttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcattaaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccctagactcacgctgggcataactactttgtgacaggagcacatgcaaatgggtacaaggaggaagtcgaaacaagacgagtcgtgcaaagaatcgctgaattatgcgaagagaacggaattcactatgcaatcactacagacaacgacggacgcacacaacgccagaatttaaacaacatcatagcgaattgtaacagccatacaagagatcgtgtggacgtagcgattcacttcaaccaagcaacgtcagaaaaaggcggtgtcgaggtttggtattatgaccaatataatttagcttctaaagtgagcgaagccgtggcgggtgcgctaggtatcgcaaaccgtggagcgaagcaagggaaggaattagcggttctgaatgggacaaatgctcccgctattttaatagaggttgcattcttaaacaatccggggaatatgaaagcgtacgaggaacgatttgaagccgtttgccgcgctattgtccaaagtgttacagatgtaaatctgaagaactatcaggacttacctgtcgcaaaagcggacaaatattaccgtgtgaacttcggtgactttgacagctttgattggttagcgggtgtaaagaaacgtgtaaacgaaatattaccgggtcacggatgttggattgcttgtctagccgggaacgctcatagaatcctattaggtgactatggaagccgagaggaagcacaagccgtaattgatagattgaatcaagaatttccgggtagtggaatttggttagaagaattgtaaaataggaaagagttgtggaattgattccatgactcttttttattttgtttatatcgtgaaacatagatgtttatttgtgtcaatgggtaatatttcagccacaaccatttttggctgctttgctgtatgcttcaccagta

SEQ ID NO: 4 - protein, 734 aa, unidentified
MSTTNRKKRREIKLFTLATETRGLDGDVFRIGLFDGKQYYTGYTFADVLPVFEKYKAYDCHVYIHNLDFALSKIIAELRDYAEPTFNNSLFINGNIVTFTASHIILHDSFRLLPSSLENLCRDFDLLEGGKMDIVDYMEENNYGIYNVKNRKLNKRLTKGNFFTTVDKDDPVLCEYMEYDCRSLYKILEIVIGLSKLEVEQFINCPTTASLAKTVYKEQYKKDYKVAISTKQYNHKQLGKGLEAFIRKGYYGGRTEVFTPRIENGYHYDKNSLYPYVMKMAEMPVGYPNVLDNEEAELSFDLWKRRRYGAGFIHAKVHVPEDMYIPILPKKDYTGKLIFPVGKIEGVWTFPELALAEAEGCKIEKIESGVVFEKTAPVFREFISYFEEIKNTSKGAKRAFSKLMQNALYGKFAMQRERIMYADISERDKLEAEGHTVSEIINDMNGIRMEFLEYDGYAMAEYIQPHISAYITSIARILLFKGLKYAHEKGILAYCDTDSCATTTKFPDKMVHDKEYGKWKLEGYVIEGLYFQPKMYAEKAINTDGEYEEVLRMKGVPKWVVEEQLDYNSFRKWYLQVKRGKAEIPIYKGGERVQKFLTKSKNNIEMNELAEMHKTINFAREQKRNIDLNKNITSPLVRNDYGENKDEKSEYEFDEWYERLEEFNDDMNAVEELCMKFGKIQIPEKKQRKLYGLYKEYSSKAKAMCFSNEGLPIQDWCKKTGWDMKELLGELSFL

SEQ ID NO: 5 - DNA, 7390 bp, unidentified
  source 1..7390: mol_type = other DNA
ggatccttcctcctttaacatttccccctttgatttttagatatcactagtttggaccatttgtcatttccccctttgatttaagtgaacaagtttatccatcaactatcttaattgagttagtttgtttatccaataaactaactttatctcatcatatacaaaataaatgtttatttcaatgttttttttagaaaatttagttataatattagatatgatacttttaaatatctaattcaagcttcaaaaaacaccaacttagttcggtggataaacaaaggagtggttattattcaaattgcagatcaagctttagtaaaaaaaatgaatcaaaaattaatattagatgaaattttgaagaactcccctgtctccagggcaactctctctgagattacaggattaaacaagtctactgtctcctctcaagtaaatacactgcttgaaaaagattttatttttgaaattggggcagggcaatctagaggcggcagaagacctgtaatgcttgtttttaataagaatgcaggctactcgattggtattgatataggagtcgactatcttaacggaattctaaccgacttagaaggaaatattattctcgagaagacttctgacttgtctagttcttccgctagtgaagtaaaagagattttatttgcacttattcatggttttgtaacccatatgcctgagtccccttatggtctagtcggaataggaatttgtgttccaggccttgtagatcgtcatcagcaaattattttcatgcctaacttaaattggaatatcaaagatttgcagtttttaattgagagtgagtttaatgttccggtttttgttgaaaatgaagctaatgcaggagcatacggtgaaaaagtatttggtatgacaaaaaactatgaaaacatcgtttacatcagtattaatatcggaattggaactggacttgttattaacaacgaattgtataaaggtgttcagggtttttctggggaaatgggtcatatgacgatagattttaatggacccaaatgcagctgtggaaatcgaggctgttgggaattatatgcttctgaaaaagcgttactggcttcgctctctaaagaagaaaagaatatttctcgaaaagagattgtggaacgcgcaaataaaaatgatgtagaaatgttaaatgcacttcaaaactttggcttttatatcggaattggattaaccaatatccttaatacatttgatatagaagctgttatcttgagaaatcatataattgaatctcatcccattgttttaaatacgattaaaaacgaagtttcttctagagtccattctcatttagacaataaatgtgaactattgccttcttcgttaggaaaaaatgcacctgctttaggagcggtttctatcgttattgattcttttttaagtgttacccctataagttaggagctcctttattacaaaacgcctgcgcaataacgcaggcgttctgtgacattaacttcgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaattaattccttaaggaacgtacagacggcttaaaagcctttaaaaacgtttttaaggggtttgtagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctcggatccggcgcgcccctccttgacactgaatttagcatgtgatataattaacttaatattctacccaagcttataaaagagcactgttgggcgtgagtggaggcgccggaaaaaagcatcgaaaaaaggaggaaaaaaaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtagatctctgcagtcgcgatgattaattaattcagaacgctcggttgccgccgggcgttttttatgcactgacaataaaaagattcttgttttcaagaacttgtcatttgtatagtttttttatattgtagttgttctattttaatcaaatgttagcgtgatttatattttttttcgccgcatccaatcagttcagccatcggctcgcatgccggtccaggtacattggcgatattctttttaagaaaatgagcaaacaaaaacagtcagactctgtgtcctgactgtttttgtttgctcatagaatgccgacagcctcatacgccttttcgactgaagctgttaaaacacctcgccctcgtcaacaacctcatactccgcttccacagctggcgcagccatttcttcctccatccaagcgcgagcacggcgagcttgctgctcggtcatagagtgcatgccttcagcgcccaccgcaccacaaacagcagtcataccctcagccggagtgatgccgaagtgttcgcaatatggcttgaagagttcgcggattggctggagatccactggttgctccgcctgagcgcgcacctgttctggctgttgcgcttgatccatttcctccgcagcgtacatgccctgaaaagtgtccgggaacgcgaggcgccaagcagcagccttcgcgcatttttcgatcataacacccggcattttcgcccaattagatttgccggtggaatagtcgtcgagcgccacctcagcatacgcagtctcgcggccatccttaaagcgaacttccgcccaaccaccgatgagctcctcacccgcctttttgtagcacgcagcaccacgtttatgcacaacatcaccgtcacgcagaacaaccacaccagatttgataccatcatagttagcatcacgacacgcgcgacggttaaaaacttgataggaagtaatcatagaagctggagcaccgccgtatttcaccaggtaagcgtctttcacaaacggattcagacgttgagtctgacagagttgcagaaagaggcccacctctttttcagtagcgttctcacagatataacggcgaacatcgtctggagtaacttgaaccgccaggccagagtcgtcagtgaacttcacgatttggttcatttttatcacctcctttctgccaagctttcatcattatcagaacggaatgtcatcatcaaagtccatcggcggctcgttagacggcgctgccggagcggactgctgcgggcgagactgcgcgccgccgctgaactgattgccaccctgcggctgctgaggctgaccccaaccgccctgcggctgaccaccaccgatattgccacctgccggagcgccaccaccctgacgaccacccagcatctgcatggtgccgccaacgttcaccacgacttctgtggtgtagcgatcctgaccggattgatcggtccatttacgggtacgcagctgaccttcgatataaacctgagaacctttacgcagatattcgctcgccacttctgccagtttgccgaacagcacaacgcggtgccattcagtctgttctttcatctcgccggtcgctttatcacgccaggattcggaagtagccagcgtaatgttggcaactgcgccaccatttggcatgtagcgtacttccgggtcctgacccagattaccaacgagaataaccttgtttacgcctctgctggccatgtgtacattcctcctttacgtttcacctccttttcatcgccattgctccccaaatacaaaaccaatttcagccagtgcctcgtccattttttcgatgaactccggcacgatctcgtcaaaactcgccatgtacttttcatcccgctcaatcacgacataatgcaggccttcacgcttcatacgcgggtcatagttggcaaagtaccaggcattttttcgcgtcacccacatgctgtactgcacctgggccatgtaagctgactttatggcctcgaaaccaccgagccggaacttcatgaaatcccgggaggtaaacgggcatttcagttcaaggccgttgccgtcactgcataaaccatcgggagagcaggcggtacgcatactttcgtcgcgatagatgatcggggattcagtaacattcacgccggaagtgaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcat

SEQ ID NO: 6 - DNA, 4660 bp, unidentified
  source 1..4660: mol_type = other DNA
aaaattttctaaaattaactattgacaatttggaaaatttattggaatttccaaaaggaaaagtgtgacaagatattccaagggtgctataataggaaatgagttgaaaacgaacacgcgttttatagatgttcggtttttgtaacataactgtaacataaaaacatttgcattactggtaactttcgggtaatattggtagcagagagggacaaacgggacaaatcgagacacgatacatactctctatacaagtgaaaagaaggtggtgcatatgtaatatgagtaaactactgactgcgcaagaggttgcagacttattgcgtgttcataaaaagactatccatagaatgatacattctggaaagttagacgcttctaaggtagcaaacaaattccttattaaagaggaagatgcaaaaggactattggaaaataaaaataaaatggacaattaataggaggaatattcaatggaaaacaatactaaacaattaaataatgaggttaacgaaatcgcggtagtggacgagcatgtaaatacaaatgacatcgtaacagtggacaaggtgtcaccttcaatctttgaagcggacggagcgaaggatgtattcttctcatctattcaaagtaaagatcgtaaatcagctatcaaagtttataatgctattaactctagtgagaatgctctagcagatcatagaggtgaagtattagaaatcacggacatggttgctcatgctattactctacaagatgaattgacaaaagaagatgtggatgcattacgtgttgttttaattgataagaacggtaaagcataccacgcggtgtcacaaggggtcgtatcatctattcaaaagattattagtattgtaggtccagcaccttggaacgagcctttagaaattgttccggcagaagtgaaaacacgtaaaggatttaaaacattaactcttcaactacaaggctaatttttgtaacaaatgaatgagggtgcacattgtgtatcctcttttttaaaacataaaaggatggtgcggtcgtgaacgctattaaaaacttagttcaagtatctattcggacaaatgaacgagaactggaatttgtctcttttaatggaatggtgcatttgaagcaaaagttagaagaagatgaaacatccgtcgtgttaacaaaagaagagttagacatggttgcagctatgtctaattttgataatgtgatagccaactttatgaactctattacagaaaagaaagccatgtaagccaaagacctgtgacctagttgttacgggtcacatggctttataagtaggtgatacaatggcaaataaacggttaaagaagaaattagaaacgaaacgtaaaaagtcgttacttgtttcagaaggttatagtaagaaagaaacaaagaagctgaaaggtagagagttagaaactgtttataaaaagaaagcgcataataggaaaaatagagagcgtgctagggaaatagcaaatctagcaagacagtggggactttcaccgagtaagtttaattcatggaaaaagttacttccggaaatagagagaataaaaaaagaacaagatagggaagcaccttttctagtaatatactatcaggactttacaggtgagacagacagtaagtttatatatgattttaaaaagagaaacaatacaagatcacgtagccaaattactaggtctataataggttggttgcagaacgcccagaacaaactatttttaggaagggttgctatgcggattgtcccaaaacgtgatgtatctaaaaccaatacactttggaagaatcacggctatgtaaagatatatgagggacaaggaaaagaacttacaaaactattaacagcaatagaaacgataatggttggtgtttacgacgtaaaagaccgtgacaagtatttaaagcagttattaaataatcttcgttccttaccatataaacaagcacataggaacgctaatgagattcagaagatatatgatactaaatcatatacgaaagaaagttgggataacgatgagtactactaatagaaaaaagcgtagagagattaaactctttacgctagatactgaaacacgtggtttggacggcgacgtgtttcgaattggactattcgatggaaaacaatactatactggctatacgtttgcagacgtattaccagtgtttgaaaagtacaaagcatatgactgtcatgtatatatacataacctagacttcgacctttccaagatcatagcagagttacgtgattatgctgaaccaacatttaataactcgctatttatcaatggtaatatagtaacgtttaccgcttctcatattatactacatgatagcttccgattgctaccgtcttcacttgaaaacttatgtagagatttcgaccttttagaaggtgggaagatggatattgtcgattatatggaagaaaataattacggtatttataatgtaaaaaaccgtaaattgaacaagcgacttacaaaaggtaatttctttacaacagtagataaagatgaccctgtcctatgtgagtacatggaatatgattgtcgtagtttatataaaatcttggaaattgtaataggcctatcaaaattagaagttgaacaatttattaattgtccgactacagcttccctagctaaaacagtttataaagaacagtataagaaggattataaagttgctatttctacaaaacaatataatcataaacaactaggaaaaggtttagaggcatttattagaaaaggctactatggaggaagaaccgaagtttttacaccaagaatagaaaacggataccactatgataaaaatagtttatatccatatgttatgaaaatggctgaaatgccagtcggatacccgaatgtactggataatgaagaagcagaactttcttttgatctatggaaaagaagaagatatggagcaggctttatacatgcgaaggtacatgtacccgaagatatgtatattcctatcttacctaaaaaagactatacaggtaaattgattttcccagttggtaaaatagaaggtgtttggacatttccggaactagctttagcagaagcggaaggttgcaaaatagaaaaaatcgaaagcggtgttgtatttgagaaaacagcacctgtctttcgtgaatttatttcgtattttgaggaaataaaaaatacaagtaaaggtgctaaacgagccttttcaaaacttatgcagaatgcgttgtatggaaaatttgcaatgcagcgggaacgaatcatgtatgctgatatttctgagagagataaacttgaagcagaaggacatacagtaagcgagatcatatatgatatgaatggaattcgaatggaatttttagagtatgacggatatgctatggctgaatatattcaaccacatatttcagcatatattacttctattgcaagaattctactatttaaaggattgaagtatgcgcatgaaaaaggaattttggcttattgtgatacggacagttgtgcaacaactacgaaatttcctgataaaatggttcacgataaagagtatggaaagtggaagttagaagggtatgttattgaaggactctatttccaaccaaaaatgtatgcggaaaaagctattaataccgatggtgaatatgaggaagtattacgaatgaaaggtgttccgaaatgggttgtagaggaacaactagactacaattcatttcgtaaatggtatttacaagtaaaacgtgggaaagccgaaattcctatttataaaggcggggaacgtgttcaaaaattcttgacgaaatctaaaaataatatagaaatgaacgaactagcggaaatgcataaaacaattaattttgctagggaacaaaaaagaaacattgatctaaataaaaatattacctcacctctagttcgtaatgactatggggaaaataaggacgaaaagtcagagtatgagtttgacgaatggtatgaaagattagaagagtttaacgatgatatgaatgcggtagaggaactttgtatgaagtttggaaagatacaaataccagagaaaaagcaaaggaagttatacggactttataaagagtattcttcaaaagcgaaagctatgtgtttttcaaatgagggacttccgattcaagattggtgtaaaaagacaggatgggacatgaaagaactattaggcgaattaagtttcttataatagggagtgacgaaaaatgttgacgccaagggaacaggatacgttagaatgtataagcgggtatatggaagagtttgggtttgcaccgagtgtgcgagatatggcgagtcgaatgtacgtaagtcataagacagcacatcgctatttaatgcaattggaaagtaaaggacgaattaggagagtgcatcaaagacctagagccatccagttgttataaggaggggaaaggtatgcgtgacaaattgctcgactttatcatcgaactatcacaatctagcaaacaagtcgtaagcaagtcgtatgtcatcgacaggttaatgcaagtaacaaaagaagactacaaggaactagaaaagaaaacagaaaatgcggaaaaggactagcaaagttgctagtctttttt

SEQ ID NO: 7 - DNA, 73 bp, unidentified
  source 1..73: mol_type = other DNA
attatgtacctctactagcctattaaaatatttacctattgacacgtaataacatttatgaaatatgatatac

SEQ ID NO: 8 - DNA, 74 bp, unidentified
  source 1..74: mol_type = other DNA
tatatcgtgaaacatagatgtttatttgtgtcaatgggtaatattggtaaaagtgctagtagggatacataata

SEQ ID NO: 9 - DNA, 327 bp, unidentified
  source 1..327: mol_type = other DNA
tttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgacaaatggtccaaactagtgatatctaaaaatcaaagggggaaatgttaaaggaggaaggatcc

SEQ ID NO: 10 - DNA, 8081 bp, unidentified
  source 1..8081: mol_type = other DNA
ctccaaacaactttacacgtaattcgctaattcttttcaaatcattcaattgtctttctgtcagtttcaaattgttaccctcactttctatcaatctgctcctacacctccgttacctcgccgaccgtacgctcttctcactcattccatttcgtttgccatcagttcacgcgccgtcgtgtcctttgagagtccactccgtcaccttaccttctgtctgcacttcacttcattcatgtcgctcccttaagtcgtctacttccggctccgtcgaatacacttaataaataagtgcccatttacacagttaactgccatgggtttcctttcagtcatacaacgttcacccaagtgaataataattacaactcatacgattacaaaagccgtttcctaattgttcagggacccatgcacggatattctaaatgagtaggtggcgggcaaacttcagccacaccaactacgcatttagtttatcgtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcgaagttaatgtcacagaacgcctgcgttattgcgcaggcgttttgtaataaaggagctcctaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgacaaatggtccaaactagtgatatctaaaaatcaaagggggaaatgttaaaggaggaaggatccatgagtactactaatagaaaaaagcgtagagagattaaactctttacgctagatactgaaacacgtggtttggacggcgacgtgtttcgaattggactattcgatggaaaacaatactatactggctatacgtttgcagacgtattaccagtgtttgaaaagtacaaagcatatgactgtcatgtatatatacataacctagacttcgacctttccaagatcatagcagagttacgtgattatgctgaaccaacatttaataactcgctatttatcaatggtaatatagtaacgtttaccgcttctcatattatactacatgatagcttccgattgctaccgtcttcacttgaaaacttatgtagagatttcgaccttttagaaggtgggaagatggatattgtcgattatatggaagaaaataattacggtatttataatgtaaaaaaccgtaaattgaacaagcgacttacaaaaggtaatttctttacaacagtagataaagatgaccctgtcctatgtgagtacatggaatatgattgtcgtagtttatataaaatcttggaaattgtaataggcctatcaaaattagaagttgaacaatttattaattgtccgactacagcttccctagctaaaacagtttataaagaacagtataagaaggattataaagttgctatttctacaaaacaatataatcataaacaactaggaaaaggtttagaggcatttattagaaaaggctactatggaggaagaaccgaagtttttacaccaagaatagaaaacggataccactatgataaaaatagtttatatccatatgttatgaaaatggctgaaatgccagtcggatacccgaatgtactggataatgaagaagcagaactttcttttgatctatggaaaagaagaagatatggagcaggctttatacatgcgaaggtacatgtacccgaagatatgtatattcctatcttacctaaaaaagactatacaggtaaattgattttcccagttggtaaaatagaaggtgtttggacatttccggaactagctttagcagaagcggaaggttgcaaaatagaaaaaatcgaaagcggtgttgtatttgagaaaacagcacctgtctttcgtgaatttatttcgtattttgaggaaataaaaaatacaagtaaaggtgctaaacgagccttttcaaaacttatgcagaatgcgttgtatggaaaatttgcaatgcagcgggaacgaatcatgtatgctgatatttctgagagagataaacttgaagcagaaggacatacagtaagcgagatcatatatgatatgaatggaattcgaatggaatttttagagtatgacggatatgctatggctgaatatattcaaccacatatttcagcatatattacttctattgcaagaattctactatttaaaggattgaagtatgcgcatgaaaaaggaattttggcttattgtgatacggacagttgtgcaacaactacgaaatttcctgataaaatggttcacgataaagagtatggaaagtggaagttagaagggtatgttattgaaggactctatttccaaccaaaaatgtatgcggaaaaagctattaataccgatggtgaatatgaggaagtattacgaatgaaaggtgttccgaaatgggttgtagaggaacaactagactacaattcatttcgtaaatggtatttacaagtaaaacgtgggaaagccgaaattcctatttataaaggcggggaacgtgttcaaaaattcttgacgaaatctaaaaataatatagaaatgaacgaactagcggaaatgcataaaacaattaattttgctagggaacaaaaaagaaacattgatctaaataaaaatattacctcacctctagttcgtaatgactatggggaaaataaggacgaaaagtcagagtatgagtttgacgaatggtatgaaagattagaagagtttaacgatgatatgaatgcggtagaggaactttgtatgaagtttggaaagatacaaataccagagaaaaagcaaaggaagttatacggactttataaagagtattcttcaaaagcgaaagctatgtgtttttcaaatgagggacttccgattcaagattggtgtaaaaagacaggatgggacatgaaagaactattaggcgaattaagtttcttataaggattgaaggatgcttaggaagacgagttattaatagctgaataagaacggtgctctcggatccggcgcgcccctccttgacactgaatttagcatgtgatataattaacttaatattctacccaagcttataaaagagcactgttgggcgtgagtggaggcgccggaaaaaagcatcgaaaaaaggaggaaaaaaaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtagatctctgcagtcgcgatgattaattaattcagaacgctcggttgccgccgggcgttttttatgcactgacaataaaaagattcttgttttcaagaacttgtcatttgtatagtttttttatattgtagttgttctattttaatcaaatgttagcgtgatttatattttttttcgcctcgacatcatctgcccagatgcggatgtgcatctcctaatcaataagactacattttcgaaacataaccgttacttcaactcaaacatattccctttttgattcaattgttgctgcgttacattttcaaacatcatactcaacatttgttgcatctttggcatttgttccaacataaccttttgattcacataattctctaaagctaaatgaactaaagtgcttttcggtacaccactttctttgctatactcgtccaaccaatcatttaactttttactgatacgtgtattaacacgaaccatttcactcatctaatcacctcttgtatatcgtactacataaatataacatcagtttaaacacaatatcaacactaagatacaccagaacaatgtaaaagtgggactctatccgctattaatataaacaagttaagaaaaacaaaaagagagcatcatattgacaccctcttgacacacagagaagcacacgctaatatcataaacgagtatagttcttaatattttctaatatagataaccaacagtcctgtagaatggggtccaccgccaagtgatccatttcttcaagggctttttttatactctctttagttctacgagggaactctttcatatccatagttccattagaataacaaatcaaacaagcttttaatgcccctttaacattattcaaattaaaaatcgagaatgtataagattcatcaatactaaacggaggactcttccttatttcgtctaccactacaccgtcagagtctctccaactatactcaatacgagtcaaattctcttcttctatcattcttttaaaatgattatataactcctttttcttatcatatatcttcaaatatccatctttatttctatttccataataaacagttcctttatgtctatcctgttgctttcctgttttactataagacacaatatcactcatatgacgctctatatcaaaagcaatatccataccataaaccactcgcttattagacttcaaaactgcatttaacgtatgaaaaacggtttcaaacactttttcataactatctttttgcaactcgttattttcaaatttagaagggttaaaatcaactttcatatcatagctatctttttccttttccgaattatgtttccaccctatataaactgcaccttcaccatatccaatatgtataactctatgataatctctaacagacgtcccatgttctgacctaataggtgtattcggtaaaaaaatctgctttgacgatattttagttaaaacatcactaaattgtttctctgttacatctttatacttaatcattagcctatccaaataaacttgcataaaatctatcccgctccttattttttattaaacatgtcattttgtacacgattttcggctattagaatgtccgaaaatgtcttcgccacatttctacaatttacccacacattgaattatactgttcaatctctatcgtgacttcaaaaatttctaattgccctaaataagaactaatagcattatccaataaaaactcaacattctctggatcaactacactttcaccaggaa

SEQ ID NO: 11 - DNA, 24 bp, unidentified
  source 1..24: mol_type = other DNA
acggacagttgtgcaacaactacg

SEQ ID NO: 12 - DNA, 41 bp, unidentified
  source 1..41: mol_type = other DNA
gaaattgttatccgctccgtcacacgtgtgtcattttggac

SEQ ID NO: 13 - DNA, 42 bp, unidentified
  source 1..42: mol_type = other DNA
cacgtgtgacggagcggataacaatttcacacaggaaacagc

SEQ ID NO: 14 - DNA, 35 bp, unidentified
  source 1..35: mol_type = other DNA
gaacacgaactaacgccagggttttcccagtcacg

SEQ ID NO: 15 - DNA, 39 bp, unidentified
  source 1..39: mol_type = other DNA
ggaaaaccctggcgttagttcgtgttcgtgctgacttgc

SEQ ID NO: 16 - DNA, 42 bp, unidentified
  source 1..42: mol_type = other DNA
gccagtttcgtcgtttaatgccctttacctgttccaatttcg

SEQ ID NO: 17 - DNA, 43 bp, unidentified
  source 1..43: mol_type = other DNA
ggtaaagggcattaaacgacgaaactggctaaaataagtaaac

SEQ ID NO: 18 - DNA, 45 bp, unidentified
  source 1..45: mol_type = other DNA
gtagttatgcccagcgtgagtctagggacctctttagctccttgg

SEQ ID NO: 19 - DNA, 32 bp, unidentified
  source 1..32: mol_type = other DNA
cctagactcacgctgggcataactactttgtg

SEQ ID NO: 20 - DNA, 25 bp, unidentified
  source 1..25: mol_type = other DNA
caattacggcttgtgcttcctctcg

SEQ ID NO: 21 - DNA, 53 bp, unidentified
  source 1..53: mol_type = other DNA
tgttaaaggaggaaggatccatgagtactactaatagaaaaaagcgtagagag

SEQ ID NO: 22 - DNA, 54 bp, unidentified
  source 1..54: mol_type = other DNA
gcatccttcaatccttataagaaacttaattcgcctaatagttctttcatgtcc

SEQ ID NO: 23 - DNA, 38 bp, unidentified
  source 1..38: mol_type = other DNA
gtttcttataaggattgaaggatgcttaggaagacgag

SEQ ID NO: 24 - DNA, 58 bp, unidentified
  source 1..58: mol_type = other DNA
catggatccttcctcctttaacatttccccctttgatttttagatatcactagtttgg

(19) United States
(12) Patent Application Publication
(10) Pub. No.: US 2024/0093212 A1
(43) Pub. Date: Mar. 21, 2024

(54) BACTERIAL CONTINUOUS EVOLUTION SYSTEM, ORTHOGONAL ERROR-PRONE DNA POLYMERASE, AND CONTINUOUS EVOLUTION METHOD

(71) Applicant: JIANGNAN UNIVERSITY, Wuxi (CN)

(72) Inventors: Yanfeng LIU, Wuxi (CN); Jian CHEN, Wuxi (CN); Guocheng DU, Wuxi (CN); Long LIU, Wuxi (CN); Xueqin LV, Wuxi (CN); Rongzhen TIAN, Wuxi (CN); Runzhi ZHAO, Wuxi (CN)

(21) Appl. No.: 18/514,541

(22) Filed: Nov. 20, 2023

Related U.S. Application Data
(63) Continuation of application No. PCT/CN2023/092743, filed on May 8, 2022.

(30) Foreign Application Priority Data
Aug. 24, 2022 (CN) 202211021222.2

(51) Int. Cl. C12N 15/75; C12N 9/12

(52) U.S. Cl. CPC C12N 15/75; C12N 9/1252; C12Y 207/07007; C12N 2830/002

(57) ABSTRACT

The present invention provides a bacterial continuous evolution system, an orthogonal error-prone DNA polymerase, and a continuous evolution method. In the present invention, by combining an orthogonal DNA replication system and an orthogonal error-prone DNA polymerase, a continuous evolution method that includes all mutant types, enables long-DNA-fragment mutation, and is good in continuity and simple and convenient to operate is obtained. By inducing the opening and closing of DNA polymerase expression, switching between a linear plasmid error-prone mutation process and a high-fidelity replication process is realized, so as to achieve the efficient and continuous evolution of a target DNA sequence.

This application is a Continuation application of PCT/CN2023/092743, filed on May 8, 2023, which claims priority to Chinese Patent Application No. 202211021222.2, filed on Aug. 24, 2022, which is incorporated by reference for all purposes as if fully set forth herein.

A Sequence Listing XML file named “10015_0138.xml” created on Nov. 20, 2023, and having a size of 52,451 bytes, is filed concurrently with the specification. The sequence listing contained in the XML file is part of the specification and is herein incorporated by reference in its entirety.

### FIELD OF THE INVENTION

The present invention relates to the field of biotechnologies, and particularly to a bacterial continuous evolution system, an orthogonal error-prone DNA polymerase, and a continuous evolution method.

### DESCRIPTION OF THE RELATED ART

Directed evolution technology realizes the development of new gene expression elements or high-efficiency enzymes through library construction and high-throughput screening, and is currently widely used in enzyme engineering, metabolic engineering and other fields. However, the traditional directed evolution method requires the initial construction of an in-vitro library, which not only has low throughput, but also often requires the consumption of large time and cost. Therefore, many continuous evolution methods have been developed to overcome this difficulty. The key to continuous evolution is to realize random mutation of a target DNA sequence in vivo, which is not only simple to operate, but also can greatly improve the throughput of the library. However, at present, a continuous evolution method satisfying the following four conditions has not been developed in bacteria, that is, including all mutant types, realizing long-DNA-fragment mutation, having good continuity and having simple operation. The orthogonal DNA replication system previously developed in yeast can meet the four key characteristics. However, it is still a huge challenge to develop such a system in bacteria. Therefore, to provide a basis for the development of enzyme engineering, metabolic engineering and other fields, the present invention accomplishes a continuous evolution method based on an orthogonal linear gene expression vectors in bacteria, namely Bacillus thuringiensis.

### SUMMARY OF THE INVENTION

To solve the above technical problems, the present invention provides a continuous evolution method based on a bacterial orthogonal linear gene expression vector, which is obtained by combining an orthogonal DNA replication system with an orthogonal error-prone DNA polymerase and can meet four key characteristics, that is, including all mutant types, realizing the long-DNA-fragment mutation, having good continuity and simple operation. The continuous evolution method can be applied to the evolution of a target DNA sequence.

A first object of the present invention is to provide a bacterial continuous evolution system including a linear plasmid and a mutant DNA polymerase.

The linear plasmid includes a DNA replication and control gene cluster, a promoter and a target gene, in which the DNA replication and control gene cluster has a nucleotide sequence as shown in SEQ ID NO. 6.

The mutant DNA polymerase is obtained by mutation from a DNA polymerase having an amino acid sequence as shown in SEQ ID NO. 1. The mutation is selected from:
mutation of aspartate at position 18 to alanine, and mutation of aspartate at position 70 to alanine (D18A/D70A);
mutation of aspartate at position 18 to alanine, mutation of aspartate at position 70 to alanine, and mutation of tyrosine at position 442 to asparagine (D18A/D70A/Y442N);
mutation of aspartate at position 18 to alanine, mutation of aspartate at position 70 to alanine, and mutation of leucine at position 521 to serine (D18A/D70A/L521S);
mutation of aspartate at position 18 to alanine, mutation of aspartate at position 70 to alanine, and mutation of valine at position 191 to phenylalanine (D18A/D70A/V191F);
mutation of aspartate at position 18 to alanine, mutation of aspartate at position 70 to alanine, and mutation of valine at position 199 to phenylalanine (D18A/D70A/V199F); or
mutation of aspartate at position 18 to alanine, mutation of aspartate at position 70 to alanine, mutation of leucine at position 403 to lysine, mutation of methionine at position 404 to isoleucine, and mutation of glutamine at position 405 to methionine (D18A/D70A/L403K/M404I/Q405M).

Particularly, the DNA polymerase as shown in SEQ ID NO.1 has a sequence shown below:

MSTTNRKKRREIKLFTLDTETRGLDGDVFRIGLFDGKQYYTGYTFADVLP
VFEKYKAYDCHVYIHNLDFDLSKIIAELRDYAEPTFNNSLFINGNIVTFT
ASHIILHDSFRLLPSSLENLCRDFDLLEGGKMDIVDYMEENNYGIYNVKN
RKLNKRLTKGNFFTTVDKDDPVLCEYMEYDCRSLYKILEIVIGLSKLEVE
QFINCPTTASLAKTVYKEQYKKDYKVAISTKQYNHKQLGKGLEAFIRKGY
YGGRTEVFTPRIENGYHYDKNSLYPYVMKMAEMPVGYPNVLDNEEAELSF
DLWKRRRYGAGFIHAKVHVPEDMYIPILPKKDYTGKLIFPVGKIEGVWTF
PELALAEAEGCKIEKIESGVVFEKTAPVFREFISYFEEIKNTSKGAKRAF
SKLMQNALYGKFAMQRERIMYADISERDKLEAEGHTVSEIIYDMNGIRME
FLEYDGYAMAEYIQPHISAYITSIARILLFKGLKYAHEKGILAYCDTDSC
ATTTKFPDKMVHDKEYGKWKLEGYVIEGLYFQPKMYAEKAINTDGEYEEV
LRMKGVPKWVVEEQLDYNSFRKWYLQVKRGKAEIPIYKGGERVQKFLTKS
KNNIEMNELAEMHKTINFAREQKRNIDLNKNITSPLVRNDYGENKDEKSE
YEFDEWYERLEEFNDDMNAVEELCMKFGKIQIPEKKQRKLYGLYKEYSSK
AKAMCFSNEGLPIQDWCKKTGWDMKELLGELSFL.

Preferably, the linear plasmid further comprises a resistance gene terminated early by a stop codon, which provides an approach to determine the mutation rate of the linear plasmid vector. In an example of the present invention, an expression frame encoding erythromycin resistance protein terminated early by the stop codon “TAA” is used, which has a nucleotide sequence as shown in SEQ ID NO. 2.

Preferably, in an embodiment of the present invention, the linear plasmid includes a DNA replication and control gene cluster and an expression frame encoding erythromycin resistance protein early terminated by stop codon “TAA” and has a nucleotide sequence as shown in SEQ ID NO. 3.

Preferably, Further, the linear plasmid further comprises a replication origin at both ends. From 5′ end to 3′ end, the elements includes a left replication origin, a DNA replication and control gene cluster, a promoter, a target gene and a right replication origin sequentially. The left replication origin has a nucleotide sequence as shown in SEQ ID NO. 7, and the right replication origin has a nucleic sequence as shown in SEQ ID NO. 8.

Specifically, the left replication origin has a nucleotide sequence shown below:

attatgtacctctactagcctattaaaatatttacctattgacacgtaataacatttatgaaatatgatatac; and

the right replication origin has a nucleotide sequence shown below:

Tatatcgtgaaacatagatgtttatttgtgtcaatgggtaatattggtaaaagtgctagtagggatacataata.

Preferably, the linear plasmid has pBMB-ESC as a vector.

Preferably, the linear plasmid vector is derived from the genome of double-stranded linear DNA lysogenic phage GIL16.

Preferably, the linear plasmid vector is engineered by homologous recombination.

Preferably, the linear plasmid vector is replicated by the orthogonal DNA polymerase (the amino acid sequence of the wild-type polymerase is as shown in SEQ ID NO. 1) of GIL16, and the replication is orthogonal to the genome. The “orthogonal” means that the DNA polymerase for replicating the linear plasmid cannot be used to replicate the genome, and the DNA polymerase of the host cannot initiate the replication of the linear plasmid.

Preferably, the promoter on the linear plasmid is any promoter suitable for host cells, such as an inducible promoter. In one embodiment of the present invention, a xylose-inducible promoter, such as P_{xylA}, is used.

Preferably, the xylose-inducible promoter P_{xylA} has a nucleotide sequence as shown in SEQ ID NO. 9.

Preferably, the mutant DNA polymerase is controlled to express by an inducible promoter. In one embodiment of the present invention, a xylose-inducible promoter, such as P_{xylA}, is used.

Preferably, in an embodiment of the present invention, the mutant DNA polymerase is linked to a vector backbone pBMB.

A second object of the present invention is to provide a cell including the bacterial continuous evolution system.

Preferably, the bacterium is Bacillus thuringiensis, including but not limited to, Bacillus thuringiensis HD-1 (GenBank No: CP001903), Bacillus thuringiensis JW-1 (GenBank No: CP045030), and others.

A third object of the present invention is to provide an orthogonal error-prone mutant DNA polymerase, obtained by mutation from a DNA polymerase having an amino acid sequence as shown in SEQ ID NO. 1. The mutation is selected from:
mutation of aspartate at position 18 to alanine, and mutation of aspartate at position 70 to alanine (D18A/D70A, M6);
mutation of aspartate at position 18 to alanine, mutation of aspartate at position 70 to alanine, and mutation of tyrosine at position 442 to asparagine (D18A/D70A/Y442N, M17);
mutation of aspartate at position 18 to alanine, mutation of aspartate at position 70 to alanine, and mutation of leucine at position 521 to serine (D18A/D70A/L521S, M18);
mutation of aspartate at position 18 to alanine, mutation of aspartate at position 70 to alanine, and mutation of valine at position 191 to phenylalanine (D18A/D70A/V191F, M19);
mutation of aspartate at position 18 to alanine, mutation of aspartate at position 70 to alanine, and mutation of valine at position 199 to phenylalanine (D18A/D70A/V199F, M20); or
mutation of aspartate at position 18 to alanine, mutation of aspartate at position 70 to alanine, mutation of leucine at position 403 to lysine, mutation of methionine at position 404 to isoleucine, and mutation of glutamine at position 405 to methionine (D18A/D70A/L403K/M404I/Q405M, M21).

Preferably, the error-prone DNA polymerase includes three mutations D18A, D70A and Y442N (having an amino acid sequence as shown in SEQ ID NO. 4), and the mutation rate is 6.82×10⁻⁷ per generation per cell per base, which is 6,700 times the genomic mutation rate.

Preferably, the error-prone DNA polymerase is obtained by structural prediction and reasonably designed mutation of AlphaFold2.

A fourth object of the present invention is to provide a gene encoding the orthogonal error-prone mutant DNA polymerase.

A fifth object of the present invention is to provide an expression vector carrying the gene encoding the orthogonal error-prone mutant DNA polymerase.

A sixth object of the present invention is to provide a cell expressing the orthogonal error-prone mutant DNA polymerase. The cell may be a bacterial cell, a fungal cell, a plant cell, an animal cell or the like.

A seventh object of the present invention is to provide use of the bacterial continuous evolution system, the cell including the bacterial continuous evolution system, the mutant DNA polymerase, the gene encoding the mutant DNA polymerase, the expression vector carrying the gene encoding the mutant DNA polymerase, and the cell expressing the mutant DNA polymerase in the food and biological fields, particularly in the continuous evolution and error-prone replication of bacteria.

Preferably, the use includes adding an inducer to a cell culture, to realize the error-prone replication and random mutation of a target DNA sequence. The target DNA sequence includes, but is not limited to, a promoter, a ribosome binding site and a methanol utilization gene cluster.

An eighth object of the present invention is to provide a continuous evolution method based on a bacterial orthogonal error-prone DNA polymerase, including the step of introducing the linear plasmid and the mutant DNA polymerase into a cell (bacteria). The construction of a random mutation library of a target protein (encoded by a target gene) and use of directed evolution of high-throughput screening are realized by orthogonal error-prone replication of a linear plasmid by the DNA polymerase in a cell.

Preferably, the mutant DNA polymerase is regulated to express by an inducible promoter, and then induced to express by adding an inducer under culture conditions. In the evolution method of the present invention, by inducing the opening and closing of mutant DNA polymerase expression, switching between a linear plasmid error-prone mutation process and a high-fidelity replication process is realized, so as to control the continuous evolution process.

Preferably, the concentration of the inducer is 0.01-100 g/L.

By virtue of the above solution, the present invention has the following advantages.

By constructing a continuous evolution method based on a bacterial orthogonal linear gene expression vector in the present invention, the efficient continuous evolution of a target DNA sequence is realized. The present invention has the following advantages: including all mutation types, realizing the long-DNA-fragment mutation (the theoretical mutation frame length is greater than the phage genome length, that is, 15000 bp), having good continuity and simple operation. The orthogonal error-prone DNA polymerase is obtained by predicting the structure by AlphaFold2, reasonably designing, and determining the mutation rate. The mutation rate of the optimal mutant reaches 6.82×10⁻⁷ per generation per cell per base, which is 6,700 times the genomic mutation rate, and will not cause a significant increase in the genomic mutation rate.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

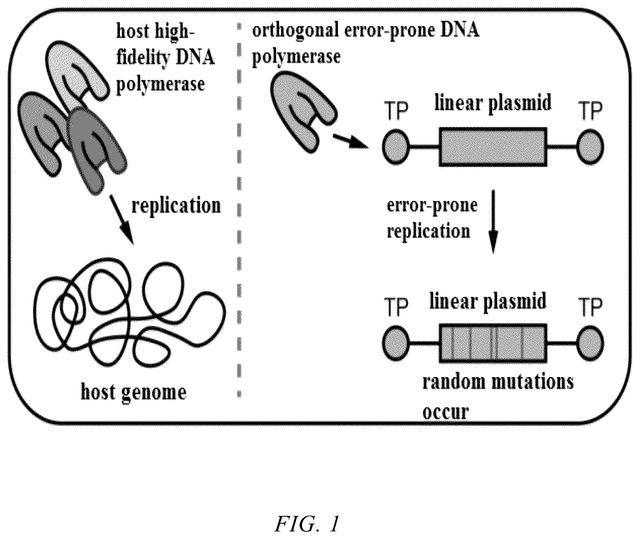

To make the disclosure of the present invention more comprehensible, the present invention will be further described in detail by way of specific embodiments of the present invention with reference the accompanying drawings, in which FIG. 1 shows a conceptual diagram of continuous evolution method based on an orthogonal linear gene expression vector.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

Materials and methods involved in the examples:
Bacillus thuringiensis HD-1 (GenBank No: CP001903).
Green fluorescent protein (GFP) under GenBank Accession No. AF324408.1.
LB medium (g/L): tryptone 10, powdery yeast 5, and NaCl 10.
SG buffer: containing sucrose 93.1 g and glycerol 150 ml in each liter.
0.1 M PBS: containing K₂HPO₄ 1.4 g and KH₂PO₄ 0.52 g, in each 100 mL.
1 M MgCl₂: containing MgCl₂·6H₂O 20.33 g, in each 100 mL.
EP buffer: containing SG buffer 1 L, 0.1 M PBS 5 mL, and 1.0 M MgCl₂ 500 μL, in each liter.

Determination method of green fluorescent protein expression: 200 μL diluted fermentation liquor per well was added to a 96-well plate, and detected by Cytation3 cell imaging microplate reader (Bio-TeK Co., Ltd), with an excitation wavelength of 488 nm, an emission wavelength of 523 nm and a gain of 60.

FIG. 1 shows a conceptual diagram of continuous evolution method based on an orthogonal linear gene expression vector.

### Example 1. Electrorotation of Bacillus thuringiensis

Preparation of competent cells: A single colony was picked up into 5 mL LB medium and activated and cultured at 30° C. overnight. Then it was transferred to fresh LB medium according to an inoculation amount of 1/100, cultured at 30° C. and 220 r/min until the OD600 was about 1.0-1.3 (about 2 hrs), and then cooled in ice bath for 10-30 min. The whole preparation process of competent cells and transformation were carried out at low temperature. After cooling, the cell suspension was centrifuged at 5000 r/5 min and 4° C. for 5 min. The cells was collected and the supernatant was discarded. Then, under the same conditions, the cells were washed twice with precooled EP buffer and once with precooled SG buffer. Finally, the cells were re-suspended in SG buffer (about 1.5 mL), to give an OD600 value of the competent cells of about 50-70. The competent cells were packaged in a centrifuge tube in 50 μL/tube and stored at −80° C. for later use; or packaged in a centrifuge tube in 500 μL/tube, and subpackaged in site immediately before use.

Electrorotation process: 1 tube of competent cells was placed on ice, and added with 3-5 μL plasmid DNA (the plasmid concentration was more than 100 ng/μL, and Escherichia coli JM110 was required to be used as the cloning host, otherwise the plasmid will be cleaved by a restriction endonuclease, resulting in transformation failure). The cells were mixed well by slightly shaking, allowed to stand in an ice bath for 10-30 min, and added to a 1 mm precooled electrorotation cup. After electrorotation at 1.25 kV, 500 μL LB medium preheated at 37° C. was quickly added. The cells were recovered and cultured at 37° C. and 220 r/min for 2 hrs, then coated on a resistant plate, and cultured overnight in an incubator at 37° C.

### Example 2. Determination of Mutation Rate of DNA Polymerase and Construction of Host

A helper plasmid pBMB-ESC (having a sequence as shown in SEQ ID NO. 5) was constructed to realize the efficient recombination of Bacillus thuringiensis HD-1. Specifically, Exo (double-stranded DNA 5′-3′ exonuclease), EcoSSB (E. Coli derived single-stranded DNA binding protein) and CspRecT (DNA annealing protein) were induced to express by xylose on this plasmid to form a single-stranded DNA by the DNA fragment and enable high-efficiency annealing in the cells.

The linear plasmid integration frame was constructed by fusion PCR. Firstly, a recombinant frame with a homologous arm length of 500-1000 bp was designed, for example, a linear plasmid (having a nucleotide sequence as shown in SEQ ID NO. 3) containing an expression frame encoding erythromycin resistance protein (having a nucleotide sequence as shown in SEQ ID NO. 2) early terminated by the stop codon “TAA”. The specific operation was as follows. Using the primers HD-TE-1F: acggacagttgtgcaacaactacg (SEQ ID NO. 11), and HD-TE-1R: gaaattgttatccgctccgtcacacgtgtgtcattttggac (SEQ ID NO. 12), the left arm was amplified. Using the primers HD-TE-2F: cacgtgtgacggagcggataacaatttcacacaggaaacagc (SEQ ID NO. 13), and HD-TE-2R: gaacacgaactaacgccagggttttcccagtcacg (SEQ ID NO. 14), the expression frame of spectinomycin resistance protein was amplified. Using the primers HD-TE-3F: ggaaaaccctggcgttagttcgtgttcgtgctgacttgc (SEQ ID NO. 15), and HD-TE-3R: gccagtttcgtcgttTaatgccctttacctgttccaatttcg (SEQ ID NO. 16), the expression frame of erythromycin antibiotic resistance protein was amplified. Using the primers HD-TE-4F: ggtaaagggcattAaacgacgaaactggctaaaataagtaaac (SEQ ID NO. 17), and HD-TE-4R: gtagttatgcccagcgtgagtctagggacctctttagctccttgg (SEQ ID NO. 18), the expression frame of erythromycin antibiotic resistance protein was amplified. The TAA stop condon was introduced. Using the primers HD-TE-5F: cctagactcacgctgggcataactactttgtg (SEQ ID NO. 19), and HD-TE-5R: caattacggcttgtgcttcctctcg (SEQ ID NO. 20), the right arm was amplified.

After purification of the obtained DNA fragment, the corresponding linear plasmid/genome integration operation was as follows. Firstly, competent cells of the strain containing pBMB-ESC plasmid were prepared. Xylose was added with a final concentration of 3% when the OD600 of the cell suspension was about 0.5, and the cells were further cultured until the OD600 was about 1.0-1.3. The other operations were the same as those in the electroporation of plasmid. During electroporation, the DNA fragment needed to be relatively simple. 5 μL DNA fragment with a concentration of above 200 ng/μL was added, and then cultured for 3 hrs. The other operations were the same as those in the electrotransformation of plasmid. Finally, by the DNA integration frame, the recombination and editing of the genome of the prophage GIL16 was realized. A linear plasmid containing the expression frame encoding erythromycin resistance protein terminated by the stop codon “TAA” was constructed. Under the same conditions, the strain containing complete erythromycin resistance gene could grow in the presence of erythromycin, and the strain containing erythromycin resistance gene early terminated by TAA stop codon could not grow in the presence of chloramphenicol. To induce the expression of DNA polymerase, the GIL16 DNA polymerase was amplified using the primers pDNAP-1F: tgTTAAAGGAGGAAGGATCCatgagtactactaatagaaaaaagcgtagagag (SEQ ID NO. 21), and pDNAP-1R: gcatccttcaatccttataagaaacttaattcgcctaatagttctttcatgtcc (SEQ ID NO. 22); and the pBMB pasmid vector containing a xylose-inducible promoter was amplified using the primers pDNAP-2F: gtttcttataaggattgaaggatgcttaggaagacgag (SEQ ID NO. 23), and HD-TE-2R: catGGATCCTTCCTCCTTTAAcatttccccctttgatttttagatatcactagtttgg (SEQ ID NO. 24). The plasmid pBMB-ODNAP (SEQ ID NO. 10) was constructed after Gibson assembly.

### Example 3. Determination of Mutation Rate of Various Orthogonal Mutant DNA Polymerase

By reasonable design, 24 mutant DNA polymerase were obtained (Table 1, the GIL16 orthogonal DNA polymerase has an amino acid sequence as shown in SEQ ID No. 1).

TABLE 1
Determination of mutation rate of various mutant DNA polymerase

| | | Mutation rate (per |
|---|---|---|
| Name of | | generation per cell |
| mutant | Reasonable mutation | per base) |
| GIL16DNA | | 2.52 × 10⁻⁹ |
| Polymerase | | |
| M1 | T19I | 3.87 × 10⁻⁹ |
| M2 | N66D | 1.57 × 10⁻⁷ |
| M3 | N66D L403R | 1.47 × 10⁻⁷ |
| M4 | H65R | 2.17 × 10⁻⁷ |
| M5 | H65R L403R | 1.26 × 10⁻⁷ |
| M6 | D18A D70A | 4.98 × 10⁻⁷ |
| M7 | Y137A | 4.69 × 10⁻⁹ |
| M8 | N66D L403K | 2.23 × 10⁻⁷ |
| M9 | L113T | 7.44 × 10⁻⁹ |
| M10 | V199F L403K L521S | 3.18 × 10⁻⁹ |
| M11 | L117V L403K Y442N | 3.38 × 10⁻⁸ |
| M12 | L113W L403K Y442N | 6.70 × 10⁻⁹ |
| M13 | L117V L403K | 3.21 × 10⁻⁸ |
| M14 | L113W L403K | 3.32 × 10⁻⁸ |
| M15 | L403K L521S | 5.57 × 10⁻⁹ |
| M16 | D18A D70A L403K | 3.34 × 10⁻⁸ |
| M17 | D18A D70A Y442N | 6.82 × 10⁻⁷ |
| M18 | D18A D70A L521S | 5.15 × 10⁻⁷ |
| M19 | D18A D70A V191F | 3.02 × 10⁻⁷ |
| M20 | D18A D70A V199F | 4.11 × 10⁻⁷ |
| M21 | D18A D70A L403K M404I Q405M | 2.50 × 10⁻⁷ |
| M22 | D18A D70A A399N F400V S401I | 1.40 × 10⁻¹⁰ |
| | L403K M404I Q405M | |
| M23 | D18A D70A L403K M404I Q405M | 1.12 × 10⁻⁸ |
| | L521S | |
| M24 | D18A D70A L403K Y442N S401I | 4.38 × 10⁻⁹ |
| | L403K M404I Q405M L521S | |

Then, the recombinant Bacillus thuringiensis constructed in Example 2 was induced by pBMB-ODNAP plasmid to express 24 additional mutants, and induced by adding 5% xylose. After inoculation at 1/1000, the cells were cultured to saturated biomass, and then diluted and coated on a plate. The proportion of resistant colonies in the total cells was counted. For each mutant, 17 replicates were set, and the fluctuation of the final result was analyzed by FALCOR tool (https://lianglab.brocku.ca/FALCOR/). The final mutation rate μ (s.p.b.) was calculated. The mutation rate is calculated by the formula μ (s.p.b.)=f/(R×C), where f is the result calculated by FALCOR, R is the unique mutant species that restores erythromycin resistance gene, and c is the copy number of plasmid. According to the sequencing result, when mutation of TAA to AAA/CAA/TTA/TAT/TAC allows the strain acquire erythromycin resistance, so R=5/3. Among the 24 mutants, M17 (having an amino acid sequence as shown in SEQ ID No. 4) has the largest mutation rate, 6.82×10⁻⁷ per generation per cell per base.

The mutation rate of wild-type orthogonal DNA polymerase was determined through the following steps. 5% xylose was added to induce the wild-type DNA polymerase to express extracellularly; and after inoculation at 1/1000, the cells were cultured to saturated biomass, then diluted and coated on a plate, and the proportion of resistant colonies in the total cells was counted. The mutation frequency of wild-type orthogonal DNA polymerase is determined to be 2.52×10⁻⁹ per generation per cell per base.

### Example 4. Control of Mutation Rate and Mutation Frequency of Target DNA by Adding Various Concentrations of Xylose

The recombinant Bacillus thuringiensis with M17 mutant constructed in Example 3 was cultured for 10 hrs in 700 μL LB medium in a 96-well deep plate at 37° C. and 750 rpm, to obtain a seed culture. Then the seed culture was transferred in an inoculation amount of 0.1% to 200 μL LB medium with various concentrations of xylose, so that the final concentration of xylose in various wells was 0.00 g/L to 50 g/L. 17 parallel replicates were set for each concentration, and cultured at 37° C. and 750 rpm for 24 hrs. Under the same conditions, the mutation rate of the control group without xylose is 2.59×10⁻⁸; and after adding 0.01, 0.05, 0.1, 0.25, 0.5, 1, 2.5, 5, 10, 30, 50 g/L xylose, the average values of mutation rate and mutation frequency are shown in Table 2 respectively.

TABLE 2
Mutation rate and mutation frequency of target DNA
after adding various concentrations of xylose

| | Xylose concentration g/L | | | | | | | | | | | |
|---|---|---|---|---|---|---|---|---|---|---|---|---|
| | 0.00 | 0.01 | 0.05 | 0.10 | 0.25 | 0.50 | 1.0 | 2.50 | 5.0 | 10.0 | 30.0 | 50.0 |
| Mutation | 0.26 | 0.43 | 0.54 | 0.47 | 0.79 | 2.31 | 4.07 | 5.68 | 6.26 | 5.36 | 5.31 | 5.32 |
| frequency | | | | | | | | | | | | |
| (×10⁻⁷) | | | | | | | | | | | | |
| Mutation | 0.09 | 0.11 | 0.14 | 0.14 | 0.24 | 0.51 | 1.01 | 1.78 | 2.22 | 2.54 | 3.26 | 2.89 |
| frequency | | | | | | | | | | | | |
| (×10⁻⁵) | | | | | | | | | | | | |

### Comparative Example 1. Determination of Genomic Mutation Rate of Strain

The method for determining the genomic mutation rate was the same as the method for determining the orthogonal DNA polymerase, except that xylose was not added, and the mutant gene selected was genomic RpoB protein. When the genome RpoB protein had the following mutations, the strain would acquire rifampin resistance: V135F (gtt-ttt), Q137R (cag-cgg), Q468R (cag-cgg), Q468K (cag-aag), Q468L (cag-ctg), H481D (cac-gac), H481P (cac-ccc), H481Y (cac-tac), H481R (cac-cgc), S486Y (tct-tat), S486F (tct-ttt), and L488S (tta-tca). Therefore, R=12/3. After the seed culture of the strain was inoculated at 1/1000, the cells were cultured to saturated biomass, and then diluted and coated on a plate. The proportion of resistant colonies in the total cells was counted. Finally, the genomic mutation frequency is 1.02×10⁻¹⁰ per generation per cell per base. Therefore, it can be calculated that the mutation rate of orthogonal error-prone DNA polymerase is 6700 times that of the genomic mutation frequency.

In addition, the genomic mutation rate of recombinant Bacillus thuringiensis with M17 mutant constructed in Example 3 was determined by the same method. The result is 1.45×10⁻¹⁰. The significance analysis shows that no significant increase in the genomic mutation rate is caused.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A bacterial continuous evolution system, comprising a linear plasmid and a mutant DNA polymerase, wherein
the linear plasmid comprises a DNA replication and control gene cluster, a promoter and a target gene, in which the DNA replication and control gene cluster has a nucleotide sequence as shown in SEQ ID NO. 6;
the mutant DNA polymerase is obtained by mutation from a DNA polymerase having an amino acid sequence as shown in SEQ ID NO. 1, wherein the mutation is selected from:
mutation of aspartate at position 18 to alanine, and mutation of aspartate at position 70 to alanine;
mutation of aspartate at position 18 to alanine, mutation of aspartate at position 70 to alanine, and mutation of tyrosine at position 442 to asparagine;
mutation of aspartate at position 18 to alanine, mutation of aspartate at position 70 to alanine, and mutation of leucine at position 521 to serine;
mutation of aspartate at position 18 to alanine, mutation of aspartate at position 70 to alanine, and mutation of valine at position 191 to phenylalanine;
mutation of aspartate at position 18 to alanine, mutation of aspartate at position 70 to alanine, and mutation of valine at position 199 to phenylalanine; or
mutation of aspartate at position 18 to alanine, mutation of aspartate at position 70 to alanine, mutation of leucine at position 403 to lysine, mutation of methionine at position 404 to isoleucine, and mutation of glutamine at position 405 to methionine.

2. The bacterial continuous evolution system according to claim 1, wherein the linear plasmid further comprises a resistance gene terminated early by a stop codon.

3. The bacterial continuous evolution system according to claim 1, wherein the mutant DNA polymerase is controlled to express by an inducible promoter.

4. A cell comprising the bacterial continuous evolution system according to claim 1.

5. A orthogonal error-prone mutant DNA polymerase, obtained by mutation from a DNA polymerase having an amino acid sequence as shown in SEQ ID NO. 1, wherein the mutation is selected from:
mutation of aspartate at position 18 to alanine, and mutation of aspartate at position 70 to alanine;
mutation of aspartate at position 18 to alanine, mutation of aspartate at position 70 to alanine, and mutation of tyrosine at position 442 to asparagine;
mutation of aspartate at position 18 to alanine, mutation of aspartate at position 70 to alanine, and mutation of leucine at position 521 to serine;
mutation of aspartate at position 18 to alanine, mutation of aspartate at position 70 to alanine, and mutation of valine at position 191 to phenylalanine;
mutation of aspartate at position 18 to alanine, mutation of aspartate at position 70 to alanine, and mutation of valine at position 199 to phenylalanine; or
mutation of aspartate at position 18 to alanine, mutation of aspartate at position 70 to alanine, mutation of leucine at position 403 to lysine, mutation of methionine at position 404 to isoleucine, and mutation of glutamine at position 405 to methionine.

6. A gene encoding the orthogonal error-prone mutant DNA polymerase according to claim 5.

7. An expression vector carrying the gene according to claim 6.

8. A cell expressing the orthogonal error-prone mutant DNA polymerase according to claim 5.

9. Use of the bacterial continuous evolution system according to claim 1 in the food or biological field.

10. A continuous evolution method based on a bacterial orthogonal error-prone DNA polymerase, comprising a step of transforming the linear plasmid and the mutant DNA polymerase in claim 1 into a cell.
